(19) United States
(12) Patent Application Publication
(10) Pub. No.: US 2019/0217071 A1
(43) Pub. Date: Jul. 18, 2019

(54) METHOD FOR REMOVING TATTOOS, AND USE OF A MICRONEEDLE PATCH IN A METHOD FOR REMOVING TATTOOS

(71) Applicant: NOLAX AG, Sempach Station (CH)

(72) Inventors: Robertino ENGEL, Suhr (CH); Christoph LANG, Nottwil (CH)

(21) Appl. No.: 16/324,606

(22) PCT Filed: Jul. 17, 2017
(86) PCT No.: PCT/EP2017/068036

§ 371 Date: Feb. 11, 2019

(30) Foreign Application Priority Data
Aug. 18, 2016 (EP) 16184672.0

(51) Int. Cl. A61M 37/00; A61Q 1/14; A61K 8/41; A61K 8/02; A61K 8/19; A61K 8/365

(52) U.S. Cl. CPC A61M 37/0015; A61Q 1/145; A61M 37/0076; A61K 8/41; A61K 2800/80; A61K 8/19; A61K 8/365; A61M 2037/0061; A61K 8/0208

(57) ABSTRACT

A method for removing tattoos, having the steps of positioning a microneedle patch (1) on a tattooed region of the skin, wherein the microneedles penetrate the tattooed skin layer; introducing, in particular by manual pressure, a solution, in particular an aqueous solution, for dissolving the color pigments into the skin through the microneedles of the patch; allowing the introduced solution to act for a duration of time ranging from 15 to 45 minutes, preferably from 20 to 40 minutes, particularly preferably from 25 to 35 minutes; and removing the color pigments, in particular extracting the color pigments using low pressure, through the microneedles (2) of the patch.

The invention relates to a method for removing tattoos, to the use of a microneedle patch in a method for removing tattoos and to a kit as claimed in the preamble of the independent claims.

Tattoos are currently removed by laser method because of the usually satisfactory results. This involves heating the skin-embedded color pigments sufficiently strongly by laser irradiation for said pigments to burst apart. Owing to the energy input, laser treatments can lead to damage to the top and surrounding skin layers. The cleavage products of the color particles that remain are then taken away by the lymphatic system. Laser irradiation is disadvantageous, since it is expensive and laborious. In the case of excessively long exposure, the skin may, however, also burn. Moreover, many sessions are required to remove a tattoo. Furthermore, a different laser wavelength must be used for each color. The cleavage products are harmful to health, since they can absolutely be carcinogenic. Furthermore, the cleavage products of the laser treatment often settle in the lymph nodes and remain in the body.

As an alternative to laser tattoo removal, there are methods using liquid removal. This concerns chemical solutions, usually acids, for example lactic acid, which are introduced onto or into the skin. These substances cause skin inflammation with wound formation. The body exudes, via said wound, a wound secretion together with the skin-embedded tattoo pigments. These chemical methods are aggressive for the treated skin areas and have the risk of infections and further wound-healing problems as well as scarring in the case of improper wound care.

Chemical removers are, for example, introduced into the skin by means of conventional tattoo machines or applied to the skin as a cream. Introduction by means of a tattoo machine is expensive, laborious and damages the skin. Application as a cream usually does not lead to the desired outcome and also damages other, nontattooed skin layers because of the surface use. The epidermis is, in this connection, destroyed or removed.

In the prior art, the introduction of chemical removers into the skin by means of microneedle arrays is described. WO 2010/051551 A1 discloses the application of microneedle arrays to the skin of a patient for therapeutic and preventive treatments. Here, a tattoo can be masked by a dye introduced into the skin or disintegrated by an introduced bleaching agent.

WO 2015/027328 A1 describcs the introducLion of a composition comprising bisphosphonates into a tattooed skin area, which composition causes the tattoo to fade. The composition can be introduced into intradermal areas by means of microneedle-based injection and infusion systems.

The disadvantage of the prior-art methods is that the color pigments, the cleavage products thereof and the chemical removers remain in the skin, or in the body, and are possibly harmful.

It is therefore an object of the invention to overcome the disadvantages of the prior art. More particularly, it is an object of the invention to provide a method, by means of which tattoos are removed in a simple and cost-efficient manner, the color pigments and/or introduced chemical removers do not settle in the body, and the skin, or the body, is not additionally damaged. This is achieved by the features of the independent claims.

The invention provides a method for removing tattoos, comprising the steps of positioning a microneedle patch on a tattooed area of the skin, wherein the microneedles penetrate into the tattooed skin layer, and introducing, especially by means of manual pressure, into the skin through the microneedles of the patch an especially aqueous solution for dissolving the color pigments. Furthermore, the introduced solution acts for a period within the range from 15 to 45 min, preferably from 20 to 40 min, particularly preferably from 25 to 35 min. The color pigments are removed through the microneedles of the patch, removed especially by means of negative pressure. In this way, the introduced solution remains in the skin only for a while. Since the introduced solution and the color pigments of the tattoo are removed, they do not settle in the skin or in the body. Damage to the skin or to the body by substances which remain is minimized or completely prevented.

The method according to the invention can be carried out by personnel with no special medical expertise. Such medical expertise is not required. Thus, the method,can, for example, be provided and carried out in tattoo studios or cosmetic studios.

Dissolving the color pigments is understood here and below to mean that the color pigments are dissolved out of the cell matrix.

The microneedles are hollow in order to introduce and remove solutions. At the same time, the patch is preferably flexible, i.e., not rigid, and is thus matchable with the skin.

The term “patch” is used synonymously for array, arrangement or plaster. The patch can be self-adhesive or non-self-adhesive.

The solution for dissolving the color pigments can have a pH of <7, preferably within the range from 0.5 to 4, particularly preferably within the range from 1 to 3.5, and preferably comprise lactic acid. Alternatively, the solution can have a pH of >7, preferably within the range from 10 to 14, particularly preferably within the range from 11 to 13.5, and preferably can have a base selected from the group consisting of calcium hydroxide, sodium hydroxide, potassium hydroxide, barium hydroxide, amines, for example ethylenediamine or butylamines. Alternatively, the solution can have a pH within the range from 6.0 to 8.0, preferably within the range from 6.5 to 7.5, and an enzyme. Thus, the color pigments of the tattoo are dissolved particularly efficiently out of the cell matrix of the skin. When using an enzyme in the solution, it is also possible to use pH values outside the range of from 6.0 to 8.0, if the enzyme is functional in the selected pH.

If the solution comprises lactic acid, the lactic acid can be present in a concentration within the range from 10 to 40% by volume. The pH of the solution is thus within the acidic range and can be in particular within the range from 0.5 to 4, preferably from 1 to 3.5. Thus, the color pigments of the tattoo are dissolved particularly efficiently out of the cell matrix of the skin.

A preferred solution comprises 40% by volume of lactic acid and has a pH of from 1 to 1.5. A further preferred solution comprises 20% by volume of lactic acid and has a pH of from 2.5 to 3. Such solutions are, for example, available from Skinial®.

It the solution comprises a base, the pH of the solution is within the alkaline range and can preferably be within the range from 10 to 14, preferably from 11 to 13.5. Particularly preferably, the pH is 12.5. Preferred solutions are alkaline, preferably having a pH of 12.5.

Such solutions are, for example, available under the trade name Rejuvi Tattoo Remover. Thus, the color pigments of the tattoo are dissolved particularly efficiently out of the cell matrix of the skin.

Preferably, the solution, if it has an enzyme, has a pH within the range from 6.0 to 8.0, preferably within the range from 6.5 to 7.5.

The microneedles can have a length within the range from 200 to 2000 μm, preferably from 300 to 1700 μm, particularly preferably from 400 to 1600 μm, very particularly preferably from 500 to 1500 μm. In this way, the microneedles, when used appropriately, reach into the skin layer in which the color pigments of the tattoo are situated. Thus, the solution for dissolving the color pigments is introduced in a particularly targeted manner into tattooed skin areas Lhrough the microneedles and is removed in an efficient manner after the action.

Individual microneedles of the patch can have differing lengths. Thus, using one microneedle patch, solutions for removing the color pigments can be introduced into different skin layers and removed. This increases the efficiency of removal of a tattoo, because application and removal can occur simultaneously in different skin layers.

The microneedles preferably comprise metal, glass, silicon or plastic as material of manufacture.

To remove the color pigments, a negative pressure can be applied to the microneedles. Said negative pressure is preferably within the range from 200 to 500 mmHg.

The negative pressure is preferably applied for a period of from 1 to 10 hours, preferably from 2 to 8 hours, particularly preferably from 3 to 6 hours, and there is extraction for a corresponding length of time. Thus, the color particles of the tattoo are completely removed from the tattooed skin area.

It is also possible to remove the introduced solution containing the dissolved pigments by means of a flush liquid, preferably composed of water or an isotonic NaCl solution. The flush liquid can be introduced by means of a tube via the microneedle patch. Another tube then flushes out the flush liquid containing the solution and the dissolved pigments by means of negative pressure. The introduction and suction or flush-out of the solution can, in this connection, be done continuously, with the result that a flow arises and the solution containing the dissolved pigments is continuously extracted.

A further aspect of the invention concerns the use of a microneedle patch in a method for removing tattoos as elucidated above.

Preferably, the microneedle patch is 9 cm², 25 cm² or 100 cm² in size. The microneedle patch can be rectangular, for example 3 cm×3 cm, 5 cm×5 cm or 10 cm×10 cm. Also possible are round configurations. The microneedle patches preferably have nine microneedles within an area of 4 mm².

A further aspect of the invention concerns a kit comprising one or more microneedle patches and one or more solutions for dissolving color pigments of a tattooed area. Thus, the user can, depending on the desired use, select a solution for dissolving color pigments. Optionally, the kit can comprise instructions for use, especially for carrying out a method as elucidated above. Alternatively, the kit can comprise multiple microneedle patches containing different solutions for dissolving color pigments of a tattooed area. Thus, the user can, depending on the desired use, select a microneedle patch containing a solution for dissolving color pigments.

### EXEMPLARY EMBODIMENTS

The method according to the invention was tested on a piece of pig skin. The skin was excised from a dead pig. The pig skin was first tattooed in certain areas. 24 hours after completion of the tattooing, a microneedle patch was positioned on the tattooed pig skin, with the microneedles penetrating into the tattooed skin layer. The microneedle patch has nine microneedles per 4 mm². The microneedle patch contains, in a reservoir, a solution containing 20% by volume of lactic acid having a pH of from 2.5 to 3 for dissolving the color pigments. The reservoir is in fluid connection with the microneedles. By means of manual pressure, the lactic acid solution was introduced through the microneedles of the patch into the tattooed area of the skin. The solution acted for 30 min. To remove the color pigments, a pump was connected to the microneedle patch via a connector, in this case a tube, and a negative pressure of 350 mmHg was applied. The color pigments, which were dissolved by the action of the lactic acid, were extracted by means of the negative pressure. The extraction was done for 5 hours.

The invention will be described in more detail below on the basis of figures, where:

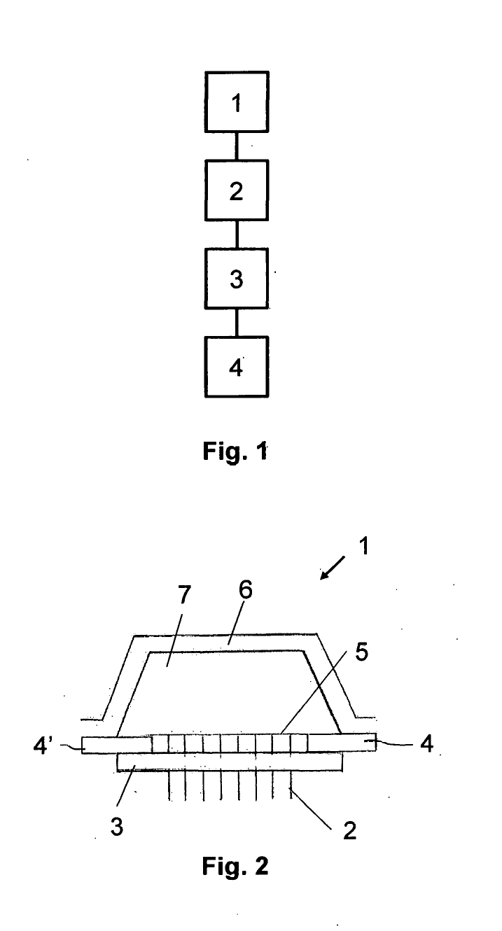

FIG. 1: shows a flow chart of the method according to the invention;

FIG. 2: shows a sectional sketch of a microneedle patch for the removal of tattoos.

FIG. 1 shows the method steps of the method according to the invention for removing tattoos. In step 1, a microneedle patch is positioned on a tattooed area of the skin, with the microneedles penetrating into the tattooed skin layer. At the same time, the microneedles penetrate skin layers situated above a tattooed skin layer and form channels into the tattooed skin layer. The tattooed skin layer is understood to mean the skin layer of a tattooed area of a skin, in which area the color pigments of the tattoo are situated.

In step 2, a solution for dissolving the color pigments is introduced into the skin through the microneedles of the patch. This is preferably done by means of manual pressure.

The introduced solution acts for a period of 30 min (step 3). Thus, the color pigments are dissolved out of the cell matrix and can then be removed.

In step 4, the color pigments, especially dissolved color pigments, are extracted through the microneedles of the patch by means of negative pressure and thus removed. For this purpose, the microneedle patch is connected to a device for generating a negative pressure. To this end, the device for generating a negative pressure is directly connected to the microneedle patch via a tube. Alternatively, the microneedle patch can be removed and a second microneedle patch for the extraction can be applied to the skin. A negative pressure of 350 mmHg is applied to the microneedle patch and extraction is carried out for a period of 5 h. Alternatively, a plaster can be applied to the microneedle patch and surrounding skin area so as to form a seal. The plaster is, in this case, connected via a tube to the device for generating a negative pressure. As elucidated above, a negative pressure can be applied and the color pigments can thus be extracted.

FIG. 2 shows schematically a microneedle patch 1 for use in the method according to the invention. The microneedle patch 1 comprises microneedles 2 which protrude through a substrate 3. The microneedles 2 and the substrate 3 form a so-called microneedle chip. The substrate 3 is, in certain areas, bonded to a hot-melt layer 4. The microneedles 2 are in contact with a membrane 5. The membrane 5 is in contact with the hot-melt layer 4. The hot-melt layer 4 is arranged on a backing layer 6. The membrane 5 and the backing layer 6 form a reservoir 7 for the accommodation of the solution for removing a tattoo. Accordingly, the microneedle patch 1 can comprise, in the reservoir 7, the solution for removing a tattoo. The membrane 5 retains the solution in the reservoir. The membrane 5 is permeable for the solution once pressure is exerted on the reservoir 7. Thus, the solution can be introduced into the skin by manual pressure.

The microneedle patch 1 is positioned on a tattooed area of the skin, for example stuck on by means of the hot-melt layer 4, with the result that the microneedles 2 pierce the upper skin layers and protrude into skin layers in which color pigments of the tattoo are situated. Owing to the hollow configuration of the microneedles 2, they form channels between the reservoir 7 and the tattooed skin layer. The solution for removal can be introduced into the tattooed area through the microneedles 2. After an action time within the region of 30 min, the color pigments, especially dissolved color pigments, are extracted via the microneedles 2 by means of negative pressure.

1-7. (canceled)

8. A method for removing tattoos from skin, the method comprising:
positioning a microneedle patch on a tattooed area of the skin, wherein the microneedles penetrate into a layer of the tattooed skin;
introducing into the skin, through the microneedles of the patch, a solution for dissolving the color pigments;
allowing action of the introduced solution for a period within a range from 15 to 45 minutes; and
removing the color pigments through the microneedles of the patch.

9. The method according to claim 8, wherein the solution for dissolving the color pigments:
has a pH of <7; or
has a pH of >7;
or has a pH within the range from 6.0 to 8.0 and an enzyme.

10. The method according to claim 8, wherein the microneedles have a length within the range from 200 to 2000 μm.

11. The method according to claim 8, wherein individual microneedles of the patch have differing lengths.

12. A kit comprising:
one or more microneedle patches; and
one or more solutions for dissolving color pigments of a tattoo.

13. The method according to claim 8, wherein the solution for dissolving the color pigments is introduced by manual pressure.

14. The method according to claim 8, wherein the color pigments are removed, by extraction, by negative pressure.

15. The method according to claim 8, wherein the solution for dissolving the color pigments has a pH within a range of from 0.5 to 4.

16. The method according to claim 8, wherein the solution for dissolving the color pigments comprises lactic acid.

17. The method according to claim 8, wherein the solution for dissolving the color pigments has a pH within a range of from 10 to 14.

18. The method according to claim 8, wherein the solution for dissolving the color pigments comprises a base selected from the group consisting of:
calcium hydroxide,
sodium hydroxide,
potassium hydroxide,
barium hydroxide,
amines, for example ethylenediamine or butylamines.

19. The method according to claim 16, wherein the lactic acid is present in a concentration within a range of from 5 to 45% by volume.

20. A kit according to claim 12, wherein the kit additionally comprising instructions for carrying out a method for removing tattoos from skin, the method comprising:
positioning a microneedle patch on a tattooed area of the skin, wherein the microneedles penetrate into a layer of the tattooed skin;
introducing into the skin, through the microneedles of the patch, a solution for dissolving the color pigments;
allowing action of the introduced solution for a period within a range from 15 to 45 minutes; and
removing the color pigments through the microneedles of the patch.
